(19) United States
(12) Patent Application Publication
(10) Pub. No.: US 2003/0143713 A1
(43) Pub. Date: Jul. 31, 2003

(54) Novel glycosidase inhibitors and their pharmacological uses, in particular for treating diabetes

(76) Inventors: Nushin Banu Helene Aghajari, Lyon (FR); Xavier Guy Robert, Lyon (FR); Richard Michel Haser, Lyon (FR)

Correspondence Address: YOUNG & THOMPSON, 745 SOUTH 23RD STREET 2ND FLOOR, ARLINGTON, VA, 22202

(21) Appl. No.: 10/168,703

(22) PCT Filed: Dec. 20, 2000
(86) PCT No.: PCT/FR00/03600

(30) Foreign Application Priority Data
Dec. 23, 1999 (FR) 99/16409

(51) Int. Cl. C12N 9/24; A61K 31/198; A61K 31/325; A61K 31/13; C08B 37/00

(52) U.S. Cl. 435/200; 514/564; 514/674; 536/53; 560/158

(57) ABSTRACT

The invention concerns the use of a polyamine derivative or a polyamine for inhibiting the active site of glycosidase enzymes intervening in the transformation of polysaccharides into sugars, in particular into glucose, in a living organism.

The invention relates to new glycosidase inhibitors and their pharmacological uses, in particular for treating diabetes.

Among the glycosyl hydrolases, some are responsible for the degradation or digestion of sugars. Some of these enzymes, such as the x-amylases, are very important in biotechnology (the detergent, derivative and the starch conversion bio-industries etc.), but also as targets for molecules of pharmacological interest, for example for the treatment of diabetes.

More precisely, the amylases are hydrolytic enzymes which are widespread in nature; and in particular they are found in animals, microbes, plants and fungi. These enzymes are involved in the degradation of sugars into oligosaccharides, such as starch and glycogen, by hydrolysis of the α-1,4 interglycosidic bonds (for the α-amylases). Barley grains contain two main α-amylase isoenzymes, AMY1 and AMY2, which are both involved in the degradation of starch to provide energy for the development of the plant embryo.

Furthermore, studies have been carried out on the resolution of 3D structures of isoenzyme 2 (AMY2) from barley grains (Crystal and molecular structure of barley α-amylase, Kadziola et al., J Mol. Biol. (1994) 239, 104-121; Molecular Structure of a barley α-amylase inhibitor complex: implications for starch binding and catalysis—Kadziola et al. J. Mol. Biol. (1998) 278, 205-217). The function of these isoenzymes is to catalyse the conversion of polysaccharides (starch, various sugars etc.) at various stages of germination, with a view to the production of sugars that can be assimilated by the plant for its physiological and energy needs. Research has made it possible to establish the detailed architecture of these proteins, as well as the precise topology of the active sites where, the reactions catalysed by these enzymes take place. One of the main objectives is to better understand the remarkable differences between the physico-chemical properties of these two isoenzymes, despite their very marked sequence homology (nearly 80% identity).

Faced with “The irresistable planetary increase of diabetes” (there will soon be 300 million diabetics, Le Monde, Apr. 19, 1999), it seems important and urgent to continue to develop lines of research in this field. Among these, the characterization, the perfecting of new inhibitors of therapeutic interest to block the action of glycosidases, constitutes one of the important approaches to the problem.

Acarbose type inhibitors (of a polysaccharide nature) have been tried and tested, in particular in the treatment of non-insulin dependant diabetes. In future they will be commercially available in many countries. Like any medicament, these molecules are not without side effects, hence the importance of exploring other lines.

The known role of spermidine (and other similar polyamines) is its vital activity in cell proliferation, growth and differentiation. It is well established that polyamines interact with DNA. Some proteins, such as the insulin receptor, protein-kinase CK2, the N-methyl-D-asparate receptor, have a specific site for the recognition of polyamine probably having a regulatory function.

To date, there are only 2 examples of protein spermidine complexes for which the 3D structures have been determined and the atomic coordinates filed.

They are:

Aminoglycoside 3-N-acetyltransferase from Serratia marcescens—the structure of which was resolved by X-ray diffraction (filed in the Protein DataBank under code 1B04). (Wolf, E.et al., Crystal Structure of a Gcn5-Related N-acetyltransferase: Serratia marcescens aminoglycoside 3-N-acetyltransferase. Cell (1998), (94)4, 439-449);

spermidine/putrescine—the structure of which was resolved by X-ray diffraction (filed in the Protein DataBank under code 1POT and 1POY) (Sugiyama S et al. The 1,8-A X-ray structure of the Escherichia coli PotD protein complexed with spermidine and the mechanism of polyamine binding. Protein Sci. (1996), 5(10), 1984-1990; Sugiyama S et al., Crystal structure of PotD, the primary receptor of the polyamine transport system in Escherichia coli. J. Biol. Chem. (1996), 271(16), 9519-9525).

One of the aspects of the invention is to propose a new class of glycosidase inhibitors, and in particular (x-amylases without the side effects of the current inhibitors, and which are non-toxic.

Another aspect of the invention is to propose new glycosidase inhibitors with a production cost which is lower than that required for preparing the currently known inhibitors.

These different aspects are covered by the invention, which in general relates to the use of a polyamine type molecule, of a polyamine derivative or of a polyamine to inhibit the active site of the glycosidases involved in the conversion of polysaccharides to sugars, in particular to glucose, in a living organism.

By “polyamine type molecule”, is designated any molecule belonging to the chemical super family of polyamines, which are molecules containing at least two amine functions.

By “polyamine derivative”, is designated any molecule belonging to the chemical super family of polyamines, but containing chemical modifications and/or grafted chemical functions not belonging to the chemical super family of polyamines.

The invention also relates to the use of a polyamine type molecule, of a polyamine derivative or of a polyamine to inhibit in vitro the active site of glycosidases used in the conversion of polysaccharides to sugars, in particular to glucose, in a living organism.

The invention relates to the revealing of a new class of α-glycosidase inhibitors, of polyamine type, and therefore of a chemical nature which is radically different from the inhibitors currently used in pharmacology for the treatment of diabetes and also useful for the treatment of other metabolic disorders, such as obesity. Representing this new class of agents is a natural polyamine, spermidine: NH₂—(CH₂)₄—NH—(CH₂)₃—NH₂.

Unexpectedly it has been found that polyamines have an inhibitory activity on glycosidases, because of their affinity vis-a-vis the enzymatic target that the glycosidases, in particular the α-amylases constitute.

The existence of a specific interaction between a spermidine type polyamine and a glycosidase, for example isoenzyme 1 of barley α-amylase (AMY 1) has in fact been shown.

It has even been observed, within the scope of the invention, that in the presence of the two types of inhibitors (spermidine and acarbose), the polyamine type inhibitor is recognised in a preferential manner by the enzymatic target, showing that it has a significantly greater affinity for the enzyme in comparison to the acarbose type inhibitors (of polysaccharidic nature) currently on the market.

Man, animals or plants are all equally designated as living organisms.

The inhibitory action mentioned above occurs equally well in vitro as in vivo, and involves an α-amylase type enzyme belonging to the α-glycosidase family which is present and operational in all living organisms.

That is why the invention relates to the use of a polyamine derivative or a polyamine for the preparation of a medicament intended for the diagnosis, the prevention or the treatment of pathologies involving metabolic disorders linked to the glycosidases, and more particularly a deregulation of the intestinal absorption of glucose, as in non-insulin dependant diabetes, obesity, hyperglycaemia, or hyperlipidemia.

The use of polyamine type molecules to inhibit α glycosidases in a competitive manner in comparison to natural substrates present in the patients to be treated, by slowing down the absorption of sugars, is a new therapeutic concept according to the invention.

According to an advantageous embodiment, the invention relates to the use of polyamines, which include at least 2 positive charges, in particular at least 3 amine functions, and if appropriate at least 1 osidic (or saccharidic) function, linear or branched, the said positive charges, in particular the said amine functions being spaced out by carbon chains the length of which is from approximately 2 carbon atoms to approximately 8 carbon atoms, in particular from approximately 3 carbon atoms to approximately 5 carbon atoms.

To clarify matters, the distance between two adjacent positive charges, in particular between two amine functions, is approximately 4 Å to approximately 7 Å.

In fact, as far as spermidine is concerned (see FIG. 1), it can be noted that the distance between the positive charges carried by two adjacent amine functions along the chain is approximately 5 Å for N6-N10 and approximately 6.5 Å for N1-N6.

This assembly advantageously forms a linear or branched chain of 7 to 19 carbon atoms, and preferably of 9 to 15 carbon atoms.

The invention also relates to a complex between a polyamine and a glycosidase enzyme, in particular glycosyl hydrolase and more particularly α-amylase, present in all living organisms and responsible for the reactions of conversion and hydrolysis of oligosaccharides and polysaccharides to simpler osidic molecules such as maltose and glucose, in which the polyamine is fixed at the level of the active site of the enzyme, in particular by hydrogen bonds involving the positive charges of the polyamine, corresponding to its amine functions, and the carboxylic functions of lateral chains of the amino acids of the above-mentioned enzyme, the number of hydrogen bonds being advantageously at least 4.

According to an advantageous embodiment, the invention relates to a crystalline complex between a polyamine and a glycosidase enzyme.

By “active site of the enzyme”, is designated the specific region of the enzyme involved in the fixation of a glucose type unit belonging to the oligosaccharide or polysaccharide fixed by the enzyme. For example, a tetrasaccharide bound to the enzyme in the active site will occupy four sub-sites.

According to an advantageous embodiment, in the complex of the invention, at least two of the sub-sites of the active site of the enzyme are involved in the bond with the above-mentioned polyamine.

By “sub-site of the active site of the enzyme”, is designated a partitioning of the active site of the enzyme corresponding under physiological conditions to the fixation of a single osidic unit of a polysaccharide.

According to another embodiment, when in the complex of the invention, glycosidase enzyme is α-amylase, in particular barley α-amylase (AMY 1), the following four amino acids of the enzyme: Glu (205), Trp (207), Asn (209), Asp (180) are involved in the bond with the polyamine.

The complex according to the invention, in particular between α-amylase and spermidine, can be characterized by at least one of the following interactions and in particular by all of the following interactions, which are hydrogen type bonds and which can be defined as indicated hereafter:

| Spermidine atoms: | AMY1 residues or water: | Distance (Å): |
|---|---|---|
| Number of the nitrogen atom | | |
| N1 | W207 Nε1 | 3.7 |
| N1 | N209 Oδ1 | 3.4 |
| N1 | Wat 1250 | 3.1 |
| N6 | E205 Oε2 | 3.7 |
| N10 | D180 Oδ1 | 2.7 |
| N10 | D180 Oδ2 | 3.7 |
| N10 | Wat 1087 | 2.8 |
| N10 | Wat 1102 | 2.9 |
| N10 | Wat 1259 | 2.8 |

In the AMY 1 residue or water column, the position of the amino acid (1 letter code) in the enzyme, as well as the position of the nitrogen or oxygen atom in the lateral chain of the amino acid is indicated.

“Wat” is an abreviation corresponding to a water molecule, in the enzyme environment, and is referred to by an arbitrary number (see FIG. 1).

The complex according to the invention, in particular between α-amylase and spermidine can be characterized by the following additional interactions:

| Additional interactions (second level): | Distances (Å): |
|---|---|
| Wat 1250 <=> W207 Nε1 | 3.6 |
| Wat 1250 <=> Wat 1637 | 3.2 |
| Wat 1087 <=> D180 Oδ1 | 3.8 |
| Wat 1087 <=> D180 Oδ2 | 3.3 |
| Wat 1087 <=> Y52 O | 3.7 |
| Wat 1087 <=> H93 Nε2 | 3.7 |
| Wat 1102 <=> D291 Oδ1 | 3.5 |
| Wat 1102 <=> D291 Oδ2 | 3.8 |
| Wat 1102 <=> H290 Nε2 | 3.5 |
| Wat 1102 <=> Wat 1259 | 2.4 |
| Wat 1259 <=> D180 Oδ1 | 3.2 |
| Wat 1259 <=> D291 Oδ1 | 3.8 |
| Wat 1259 <=> D291 Oδ2 | 2.8 |
| Wat 1259 <=> H290 Nε2 | 3.1 |
| Wat 1259 <=> R178 Nη2 | 3.3 |

By second level interaction, is designated for example those interactions that are weaker than the hydrogen bonds, for example Van der Waals interactions as well as the hydrogen bonds which do not directly participate in interactions with the polyamine.

According to another embodiment, in the complex according to the invention, the polyamine is a chain of approximately 6 to approximately 20 atoms, in particular comprising approximately 6 to 15, and advantageously approximately 6 to 10 nitrogen atoms, advantageously two primary amine functions, respectively at each of the ends of the polyamine, at least one of the amine functions being optionally substituted by a substituent chosen from, linear or cyclic polysaccharides having, approximately 1 to approximately 6 osidic units which are advantageously glucose, maltose, or cyclodextrine, and at least one of the nitrogen atoms inside the chain being optionally substituted by a substituent chosen from the linear or cyclic oligosaccharides, with 1 to 6, in particular 1 to 3 osidic units, in particular glucose, maltose or cyclodextrine.

Based on this complex and the interactions between the polyamine ligand and the enzymatic target, other complexing models can be proposed, for example with polyamines with a length varying between 6 and 20 atoms, comprising in total at least three amine functions, one at each end. One of the terminal amines (or one inside the chain) can be substituted by a substituent chosen from the linear or cyclic oligo- or polysaccharides, comprising 1 to approximately 6 osidic units, and being advantageously glucose, maltose or cyclodextrine.

In the complex of the invention, the enzyme is α-amylase and the polyamine is chosen from spermidine and its derivatives and corresponds to one of the following general formulae:

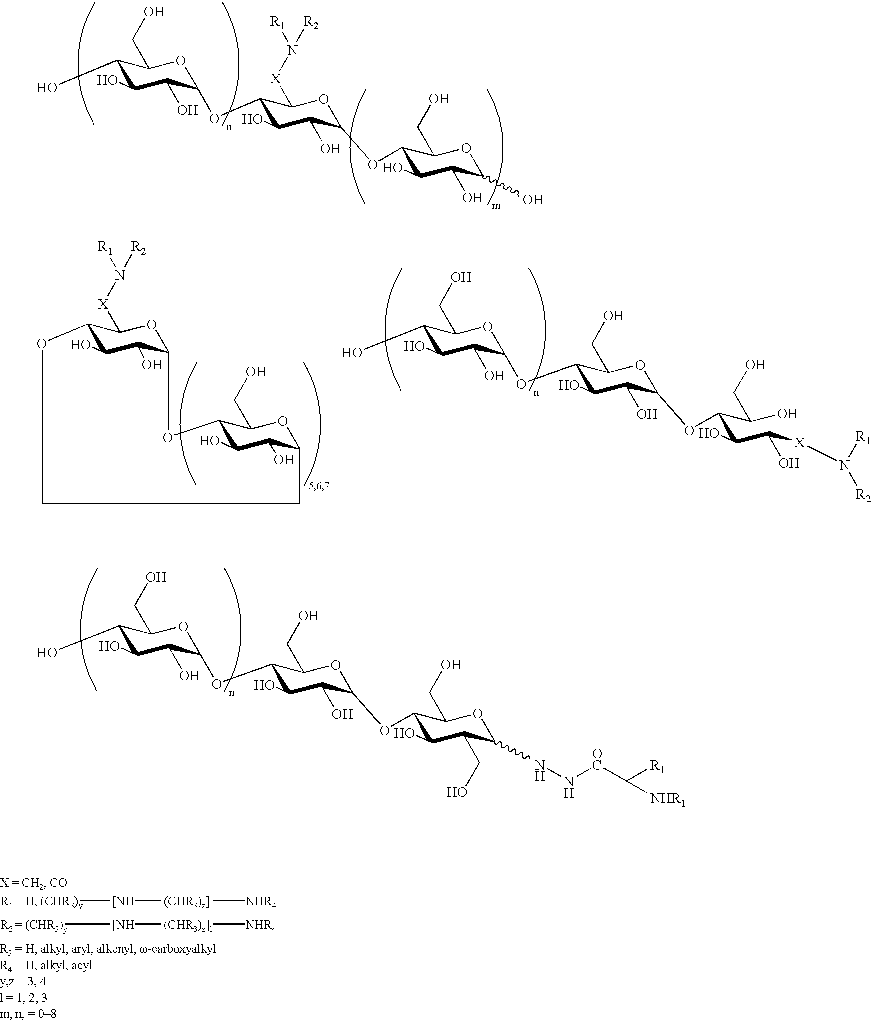

or corresponds, in an advantageous manner, to this general formula:

NH₂—(CH₂)ₘ—NH—(CH₂)ₙ—NH—R

in which m and n are comprised between 2 and 8, and in which the R group is either H, or a glucose group, or a maltooligosaccharide group, or an aryl group advantageously comprising 6 carbon atoms or an alkyl group advantageously comprising 6 carbon atoms,

or advantageously to the following formulae:

NH₂(CH₂)₃NH(CH₂)₃NH₂

NH₂(CH₂)₄NH(CH₂)₄NH₂

NH₂(CH₂)₄N(CH₃)(CH₂)₃NH₂

NH₂(CH₂)₄NH CH₂CH(CH₃)CH₂NH₂

NH₂(CH₂)₄NH(CH₂)₂CH(CH₃)NH₂

NH₂(CH₂)₃CH(CH₃)NH(CH₂)₃NH₂

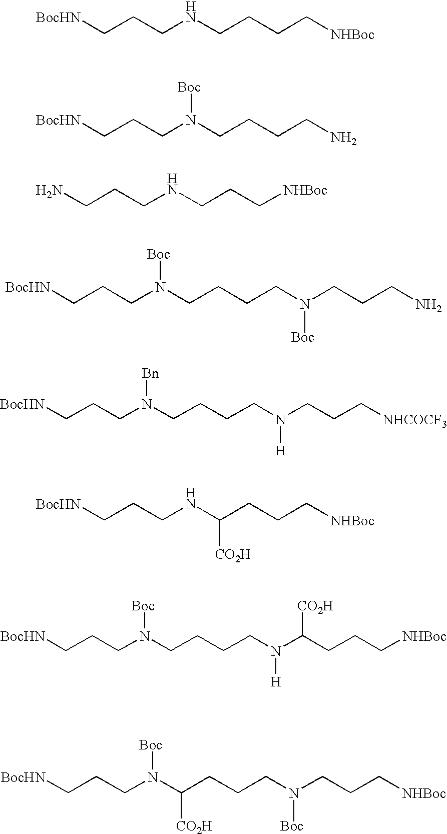

Bn=benzyl

Boc=butyloxycarbonyl,

the length of the alkyl chains between the nitrogen atoms being able to vary from approximately 2 to approximately 8 carbons, and in particular from approximately 3 to approximately 5 carbons, the alkyl chains being also able to be substituted by chemical groups preferably comprising an aminated function or derivative, the alkyl chains being also able to comprise nitrogen atoms other than those represented in the formulae above.

The complex according to the invention can be characterized by the following crystallographic characteristics:

crystalline parameters of the elementary lattice:

a=42.6 Å

b=80.6 Å

c=137.0 Å

α=β=γ=90.0°

orthorhombic crystalline system

space group P2₁2₁2₁

number of enzyme molecules per crystalline lattice of the elementary lattice=4

The invention also relates to new polyamines, capable of being used in the constitution of the complex defined above and in particular constituted by a chain of approximately 6 to approximately 20 atoms, in particular comprising approximately 6 to approximately 15, and advantageously from approximately 6 to approximately 10 nitrogen atoms, advantageously two primary amine functions, at each of the ends of the polyamine respectively, at least one of the amine functions being optionally substituted by a substituent chosen from the linear or cyclic polysaccharides having approximately 1 to approximately 6 osidic units advantageously glucose, maltose or cyclodextrine and at least one of the nitrogen atoms inside the chain being optionally substituted by a substituent chosen from the linear or cyclic oligosaccharides of 1 to 6 units, in particular from 1 to 3 osidic units, in particular glucose, maltose or cyclodextrine

on the condition that the polyamine differs from the following products:

spermidine

spermine

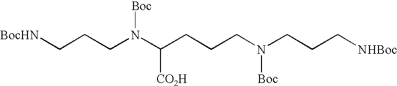

Therefore the invention also relates to the new polyamines defined above, capable of interacting with the active site of the target enzyme, according to a method of interaction similar to that observed experimentally in the crystalline state with spermidine in contact with the enzyme considered.

The invention relates in particular to the polyamine derivatives of the following general formula:

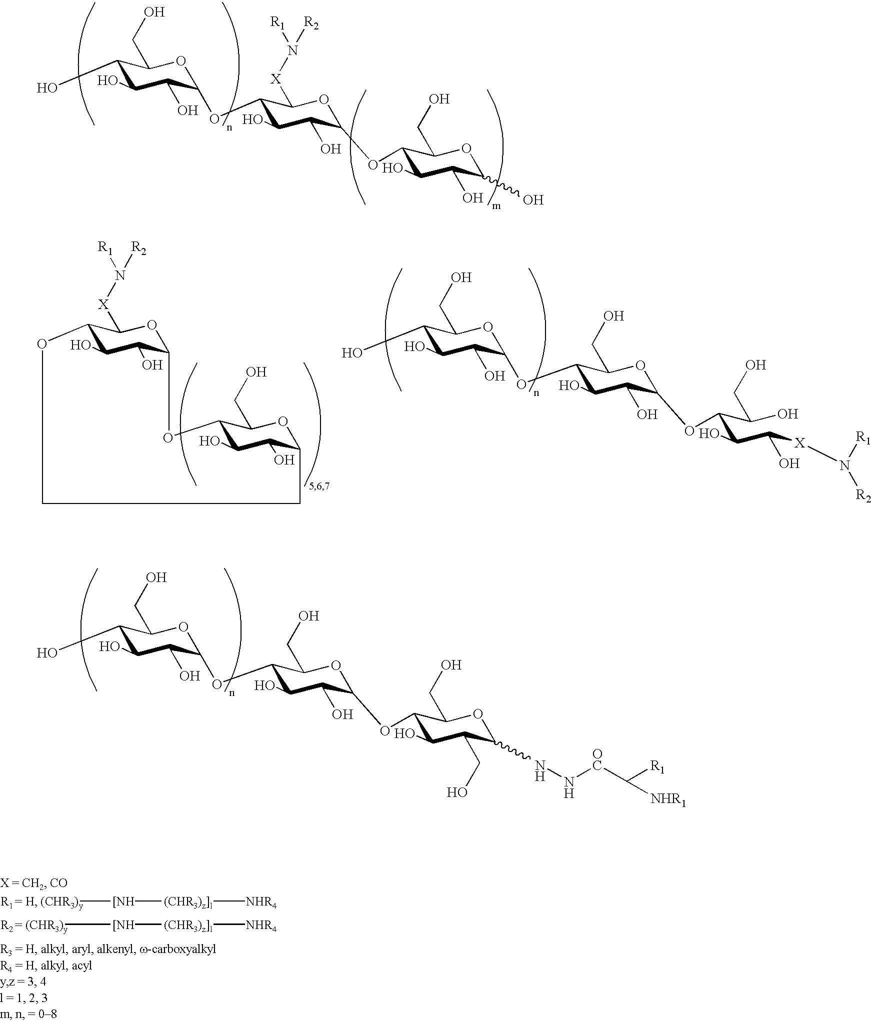

or corresponding, in an advantageous manner, to this general formula:

NH₂—(CH₂)ₘ—NH—(CH₂)ₙ—NH—R

in which m and n are comprised between 2 and 8, and in which the R group is either H, or a glucose group, a maltooligosaccharide group, or an aryl group advantageously comprising 6 carbon atoms, or an alkyl group advantageously comprising 6 carbon atoms,or to one of the following formulae:

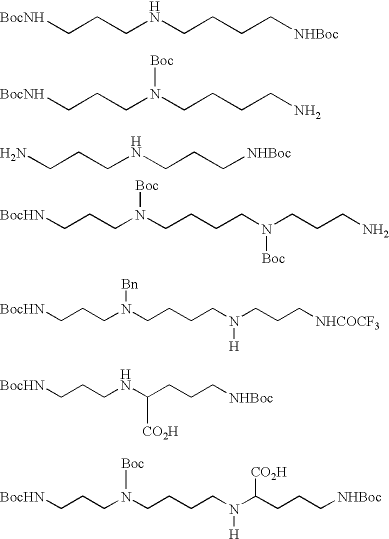

Bn=benzyl

Boc=butyloxycarbonyl,

the length of the alkyl chains between the nitrogen atoms being able to vary from approximately 2 to approximately 8 carbon atoms, in particular from approximately 3 to approximately 5 carbon atoms, the alkyl chains being also able to be substituted by chemical groups preferably comprising an amine function or derivative, the alkyl chains being also able to comprise nitrogen atoms other than those represented in the formulae above.

The polyamines of the invention, one of the aminated functions of which is substituted by one or more osidic units, and designated by glycoconjugates can be synthesized by standard methods in glycochemistry (oxidation, reductive amination, peptide coupling etc.).

In the glycoconjugate formula of the invention,

when X═CH₂, the grafting is carried out by reductive amination catalysed by sodium cyanoborohydride between the appropriately protected polyamines and oligosaccharides having an aldehyde function in position 1 or 6 and,

when X═CO, the grafting is carried out by peptide coupling between the appropriately protected polyamines and oligosaccharides having an acid function in position 1 or 6.

Synthesis of these polyamine derivatives allows very specific inhibitors to be created, targeted on a particular enzyme, in this case those of the glycosidase family involved in the reactions leading to the degradation of sugars to simpler molecules, and ultimately glucose.

### DESCRIPTION OF FIGURES

FIG. 1 represents on one hand, the numbering of the atoms of the spermidine molecule, and on the other hand the diagram of interactions between the AMY 1 residues and the spermidine molecule in the AMY 1/spermidine complex according to the invention.

FIG. 2 represents the electronic density function (determined at a resolution of 2.44 Å) of the crystal of the AMY 1/spermidine complex in the region of the active site. The extended volume of electronic density corresponds to the fixation of a spermidine molecule (NH₂—(CH₂)₃—NH—(CH₂)₄—NH₂), in the active region of the enzyme.

FIG. 3 represents the interactions of the spermidine molecule with the active site of α-amylase. The affinity of the polyamine for the enzyme is largely due to the interactions by hydrogen bonds with the three nitrogen atoms.

FIG. 4 represents the superimposition of the experimental complex: AMY1/spermidine (light <<rod>> model) and the model generated by the modelling calculations and molecular dynamics for the α-amylase of pig pancreas/spermidine (dark <<rod>> model). To simplify the diagram, the water molecules are not represented. Only the residues interacting with spermidine are represented. The nitrogens of the spermidine (linear molecule in the centre) are shown by spheres (N1, N6 and N10). The residues numbered in italics belong to AMY1.

FIG. 5 represents the superimposition of the experimental complex: AMY1/spermidine (light <<rod>> model) and the model generated by the modelling calculations and molecular dynamics for human salivary α-amylase/spermidine (dark <<rod>> model). To simplify the diagram, the water molecules are not represented. Only the residues interacting with spermidine are represented. The nitrogens of the spermidine (linear molecule in the centre) are shown by spheres (N1, N6 and N10). The residues numbered in italics belong to AMY1.

FIG. 6 represents the superimposition of the experimental complex: AMY1/spermidine (light <<rod>> model) and the model generated-by modelling calculations and molecular dynamics for human pancreas α-amylase/spermidine (dark <<rod>> model). To simplify the diagram, the water molecules are not represented. Only the residues interacting with spermidine are represented. The nitrogens of the spermidine (linear molecule in the centre) are shown by spheres (N1, N6 and N10). The residues numbered in italics belong to AMY1.

FIG. 7 represents the superimposition of 3 rings of the pseudo-tetrasaccharide acarbose inhibitor (represented by the dark <<rod>> model) and a spermidine molecule (light <<sphere and rods>> model) in their respective configuration within the active site of a barley α-amylase.

FIG. 8 represents a two-dimensional formula of the 3 rings mentioned above corresponding to an acarbose molecule after cleaving the α-1,4-interglycosidic bond leading to the loss of the glucose unit at the reducing end.

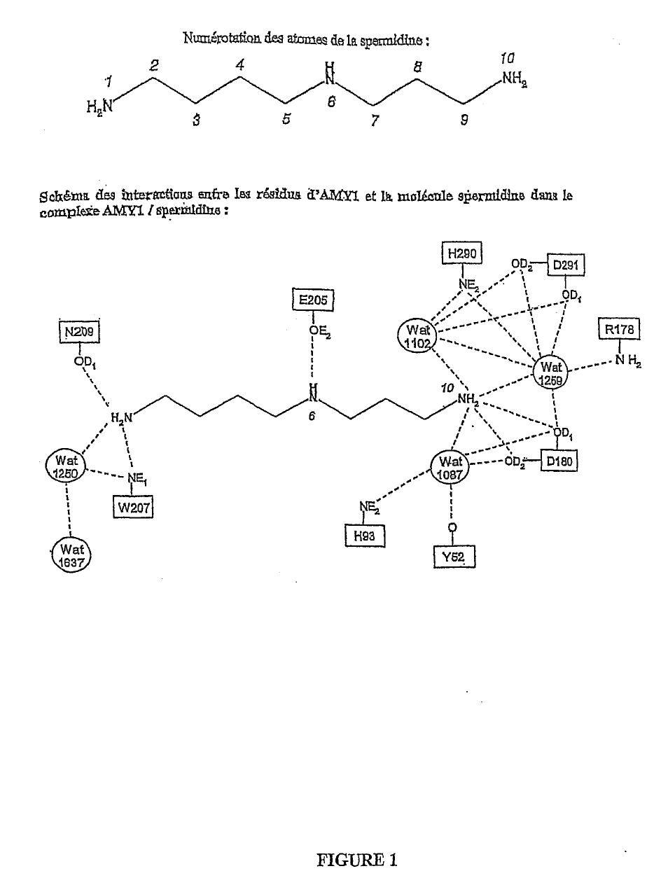

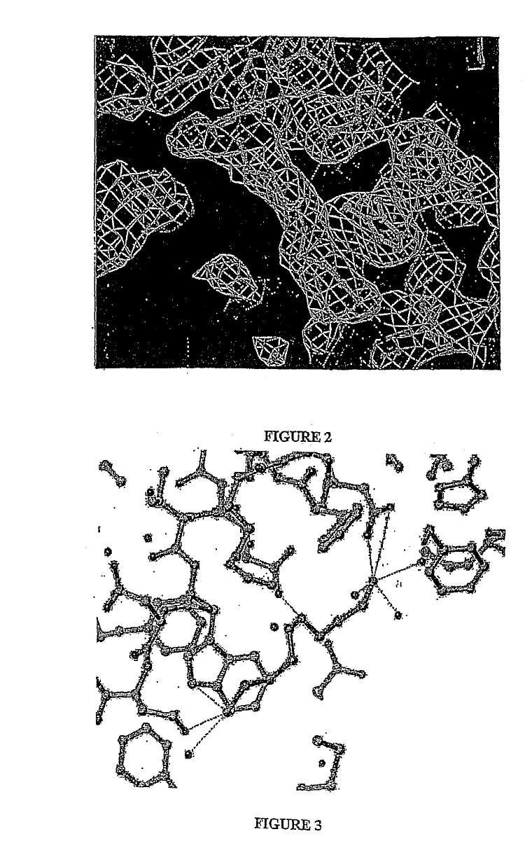

### EXAMPLES

Complexes of Spermidine or Spermidine Derivatives and α-amylase: Preparation of the Complexes and Study by Crystallography Using X-Rays

Crystallisation Conditions:

The 9 C-terminal residues of recombinant isoenzyme 1 of barley α-amylase (AMY1) were cleaved in order to obtain a C-terminal end of the same length as that of isoenzyme 2 (AMY2). The protein was then over-expressed in Pischia pastoris, purified and concentrated.

The protein preparation used for crystallisation has a concentration of 5.1 mg/ml and is in a solution of 10 mM MES (2-[N]-Morpholino ethane sulphonic acid), 100 mM CaCl₂, 0.02% NaN₃, pH 6.7.

The crystals were obtained by cocrystallisation thanks to the vapour diffusion principle using the hanging droplet technique. To do this, 2.5 μl of polyethylene glycol 8000 at 21% as a precipitating agent and 0.5 μl of a 0.1M solution of spermidine as a crystallisation additive were added to 2 μl of the protein solution described previously. This droplet was equilibrated at 19° C. against a reservoir containing 500 μl of polyethylene glycol 8000 at 21%.

The crystals were then immersed for 20 hours in a polyethylene glycol 8000 solution at 21% containing 10 mM of acarbose (a pseudo-tetrasaccharide).

Collection of the Diffraction Data:

Collection of the diffraction data was carried out using an X-ray generator (CuK_{α} radiation—wavelength 1.5418 Å) with a rotating anode (Nonius 581) operating at 40 kV and 90 mA (i.e. 3.6 kW) with a graphite monochromator coupled to a two-dimensional detector of Image Plate type (MarResearch 345) of 34.5 cm in diameter.

A set of 180 diffraction exposures (each corresponding to 1° of oscillation of the crystal) was collected at 15° C., the crystal being capillary mounted and positioned at 120 mm from the detector, with an exposure time of the crystal to the X-rays of 500s per exposure. The highest resolution of this set of data is 2.44 Å.

Characterization of the Crystal:

The integration of this data set by the DENZO software included in the marHKL package (Otwinowski, 1993 and Minor, 1993) allowed us to determine the basic. crystallographic characteristics: crystalline parameters (a=42.6 Å, b=80.6 Å, c=137.0 Å, α=β=γ=90.0°), crystalline system (orthorhombic). We then determined the space group as being a P2₁2₁2₁. The asymmetrical unit is composed of a molecule of the AMY1/spermidine complex, which corresponds to a volume of solvent in the lattice of 53%.

Determination of the 3D Structure of the AMY1/Spermidine Complex:

Subsequent processing of the data was carried out using the CCP4 suite of programs (CCP4, 1994). In this way, Scala software allowed us to show that 161737 reflections were recorded, corresponding to 17066 unique reflections with an overall R_{sym} of 13.6% and a completeness of 90.8%.

The molecular replacement method was used thanks to the AmoRe software (Navaza, 1994) using the structure of isoenzyme 2 of barley α-amylase (AMY2-accession code PDB: 1AMY) (Kadziola et al., 1994 and Kadziola et al., 1998) as a guide model because of the significant sequence homology (80%) between this initial model and our protein of interest (AMY1). Refinement phases using molecular dynamic techniques (simulated annealing—Brünger et al., 1990) were carried out with CNS software version 0.9a (Brünger et al., 1998) alternating with model manipulations. The latter, comprising the insertion of the above-mentioned molecules (water, calcium, spermidine and acarviosine) and the manual refinement of the positions of the lateral chains in the electronic density were carried out with the TURBO-FRODO software (Roussel & Cambillau, 1991). In order to do this, 2Fₒ-F_{c} and Fₒ-F_{c} electronic density maps were calculated.

In this way the refined final structure of the AMY1/spermidine complex was obtained containing 3118 non-hydrogen atoms belonging to the protein itself (i.e. 405 amino acids) and 153 water molecules, 3 calcium atoms, an acarviosine molecule (product of the degradation of the pseudo-tetrasaccharide acarbose) and a spermidine molecule.

The structure considered is characterized by an R factor of 17.2% and an R_{free} factor of 20.3% (the latter being based on 10% randomly selected diffraction data Brünger, 1992). The additional statistics concerning this structure are given in the table below.

The stereochemistry of the final model was examined with PROCHECK software (Laskowski et al., 1993) and 85.6% of the residues (non glycine or proline) are located in the most favourable regions. Finally, no residue was located in the non-authorised regions.

Statistics and collection of data and refinement
for the AMY1/spermidine complex:

| | |
|---|---|
| Resolution (Å) | 2.44 |
| Completeness of data (%) | 90.8 |
| Completeness of the data in the highest resolution shell (%) | 90.8 |
| Highest resolution shell (Å) | 2.44-2.50 |
| Total number of reflections | 161737 |
| Number of unique reflections | 17066 |
| Overall R_{sym} factor (%) (*) | 13.6 |
| Redundancy | 4.1 |
| AmoRe correlation coeficient (%) | 70.4 |
| AmoRe R factor (%) | 35.0 |
| Average R factor (%) (**) | 17.2 |
| Average R_{free} factor (%) | 20.3 |
| Total number of atoms (non hydrogen) | 3305 |
| Number of water molecules | 153 |

$$\begin{matrix}{(}^{*} ) {R}_{sym} = \frac{{\sum }_{hkl} {\sum }_{i} \vert {I ( hkl )}_{i} - ⟨ I ( hkl ) ⟩ \vert}{{\sum }_{hkl} {\sum }_{i} I ( hkl )} and \\ {(}^{**} ) R = \frac{{\sum }_{h} ‖ {F}_{obs} ( h ) \vert - k \vert {F}_{calc} ( h ) ‖}{{\sum }_{h} \vert {F}_{obs} ( h ) \vert} with k , a scaling factor .\end{matrix}$$

Significance of the Glycopolyamine Type Molecules:

One of the major proposals resulting from analysis of the AMY1/spermidine complex, is to take advantage of the existence of aromatic amino acids in the active site of the glycosidases to devise polyamines with glucose unit grafts, in order to increase the inhibition power of the derived molecule. In fact it has been established that the hydrophobic interactions between the sugar rings and the nuclei of the aromatic amino acids are an essential element in protein/carbohydrate recognition.

That is why glycopolyarmine type molecules were studied, which integrate the possibility of varied interactions with the glycosidases, on one hand due to the positive charges (NH groups) of the polyamine skeleton, and on the other hand also due to the interactions between the grafted sugars and the aromatic lateral chains of the active site. For example, the following molecules, which are spermidine—maltooligosaccharide conjugates, the synthesis protocols of which are given below, represent good candidates for α-glycosidase inhibitors:

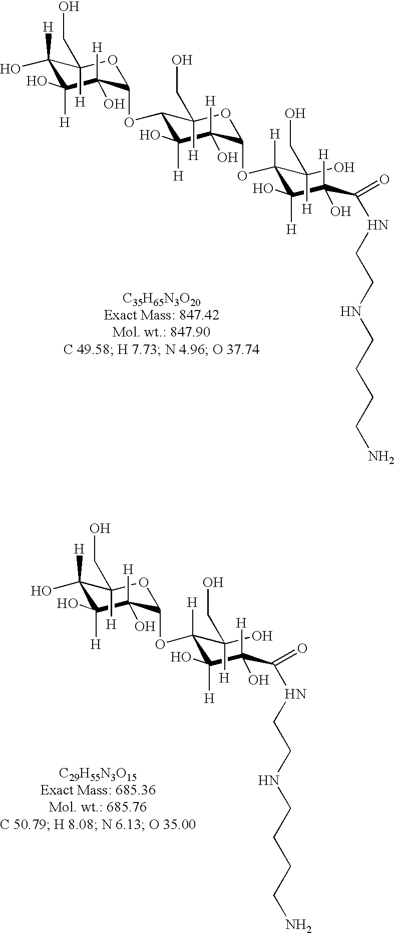

Syntheses of the Spermidine-Maltooligosaccharide Conjugates:

1—Synthesis of the Protected Spermidine Derivatives

Diprotected N⁴,N⁸ spermidine 3 is obtained in three stages from monoprotected putrescine 1. The first stage is a Michael-type addition of the putrescine onto acrylonitrile thus generating the corresponding nitrile. The secondary amine function of derivative 2 is then protected by a tert-butyloxycarbonyl group. Finally, the hydrogenation of the nitrile group in the presence of Raney Nickel in a solution of ethanolic ammonia leads to diprotected spermidine 3 (Humora et al., 1979).

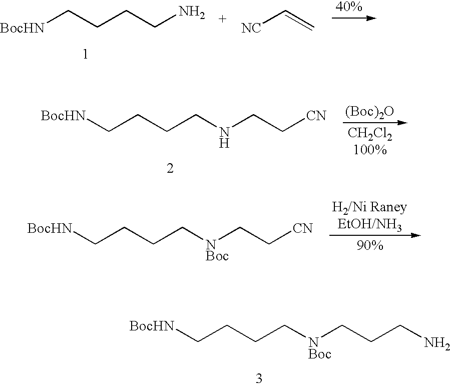

Diprotected N¹,N⁴ spermidine 4 is obtained in three stages from putrescine. The first stage is alkylation by 4-chloro-butan-1-ol, followed by protection of the two amine functions with tert-butyloxycarbonyl groups. After conversion to mesylate, introduction of an N₃ and reduction, the expected compound is obtained (Levchine et al., 1994).

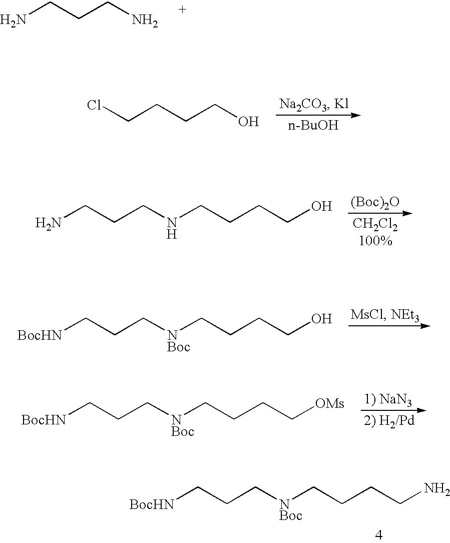

Finally, diprotected N¹,N⁸-spermidine 5 is obtained directly from spermidine by treatment with tert-butyloxycarbonyloxyimino-2-phenylacetonitrile (Boc-on) for 1 hour at 0° C. (Hesse et al., 1996).

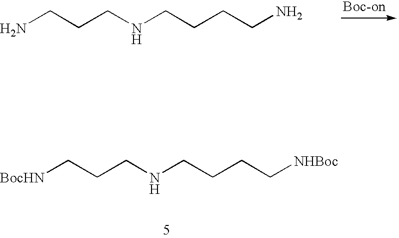

2—Oxidation of Oligosaccharides

Oligosaccharide 6 (35 mmol) is placed in solution in a water-methanol mixture (36 mL, [1:3]), then an iodine solution (17 g) in methanol (240 mL) is added and the reaction medium is heated at 40° C. A solution of potash (16 g) in methanol (400 mL) is then added and the reaction mixture stirred vigorously at 40° C. for 35 minutes. At the end of this period, the disappearance of colouring and the appearance of a white-yellow precipitate are observed. The reaction mixture is then cooled down in an ice bath and the suspension is filtered with a büchner, then rinsed with cold methanol then with ethyl ether. The solid collected is taken up in a minimum amount of water then precipitated again by adding methanol. The solid is then dried, taken up in water, then lyophilisation is carried out in order to produce the oxidized oligosaccharide in the form of potassium salt 7 (Kobayashi et al., 1985).

The acid salt in a water solution (60 mL) is treated with Amberlite resin IRN 77 H⁺ (15 g) for 30 minutes. The resin is then filtered and the filtrate concentrated in vacuo and coevaporated several times with methanol and ethanol successively in order to produce a solid residue which is taken up in water then lyophilisation is carried out in order to produce the corresponding lactone 8.

3—Coupling of Lactones 8 with the Protected Spermidine Derivative 3

A suspension of lactone 8 (2.1 mmol) and spermidine derivative 3 (1.5 mmol) in methanol (50 mL) is taken to reflux for 2 hours. The reaction mixture is then cooled down to ambient temperature, then silica gel is added (Geduran IF 60; 0.04-0.063 mesh) and concentration in vacuo is carried out. The resulting solid is then placed at the top of a Flash type chromatography column and the application of a chloroformn-methanol gradient (100% chlorofonn at [3:2]) makes it possible to isolate the protected spermidine conjugate 9, which after concentration is taken up in water then lyophilised.

4—Deprotection of the Spermidine Conjugate 9

The spermidine conjugate 8 (2 mmol) is treated with trifluoroacetic acid in water (30 mL, [9:1]) for 30 minutes at ambient temperature. The reaction mixture is then concentrated in vacuo and the solid is taken up in water and washed 3 times with ethyl acetate. The aqueous phase is then lyophilised in order to produce the spermidine conjugate 10.

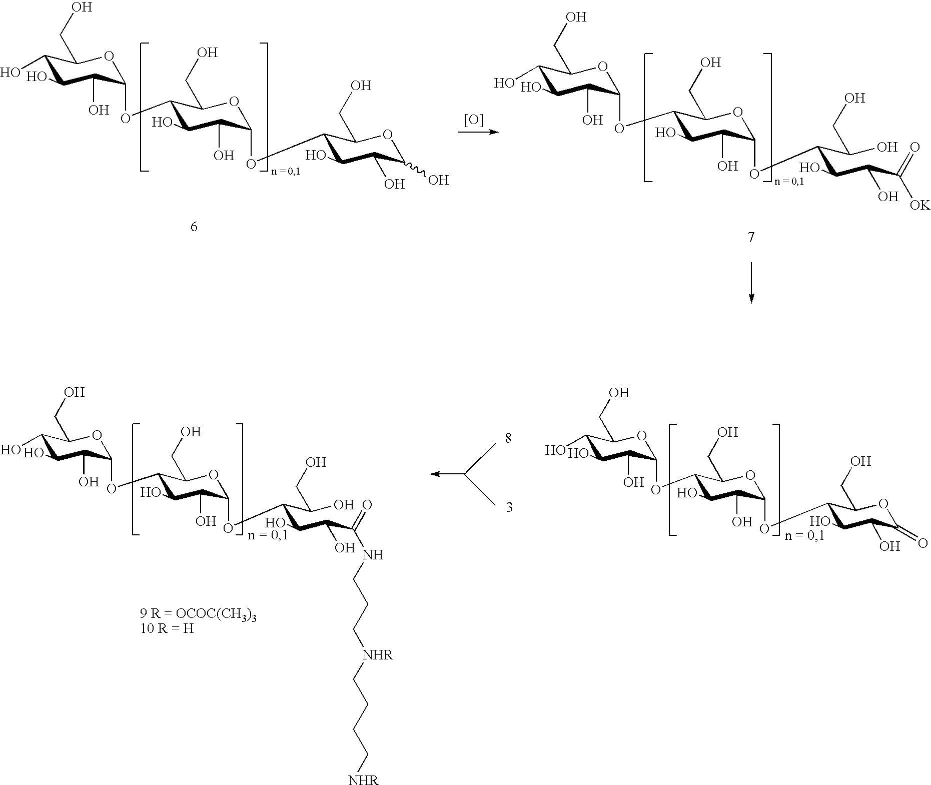

Recognition of Spermidine/α-Amylases of Various Origins by Molecular Modelling and Biocomputing Methods

Having 3D structures of other α-amylases of various origins and sets of corresponding atomic coordinates available, it was decided to extend the analysis, in particular to similar enzymes from mammals (man, pigs), in view of the considerable potential therapeutic and pharmacological implications.

1) Studied α-amylase Proteins:

With the aim of exploring the potential inhibition properties of spermidine towards other members of the α-amylase family, simulation studies of the interaction between spermidine and α-amylases of various origins were carried out by computer assisted molecular modelling methods. 3 crystalline structures of known α-amylases were therefore used for this study, and determined at high resolution by the X-ray diffraction crystallography. They are:

α-amylase from pig pancreas, resolved at 2.2 Å and complexed with acarbose, structure filed in the Protein Data Bank under the accession code 1PPI (Qian et al., 1994),

human salivary α-amylase, resolved at 1.6 Å—structure filed in the Protein Data Bank under the accession code 1SMD (Ramasubbu et al., 1996),

human pancreatic u-amylase, resolved at 1.8 Å—structure filed in the Protein Data Bank under the accession code 1HNY (Brayer et al., 1995).

2) Materials and Methods:

With the aim of carrying out these studies of interaction between the above-mentioned spermidine and α-amylases by molecular modelling methods, the 3 structures where firstly superimposed over that of the AMY1/spermidine complex. This superimposing was carried out by superimposing the a carbons of the 3 catalytic residues of each of these structures, in such a way as to match up at best with the active sites of these 4 structures.

| Name | Catalytic residues | | |
|---|---|---|---|
| Barley amylase isoenzyme | 180 | 205 | 291 |
| 1/spermidine | | | |
| Human pancreas amylase | 197 | 233 | 300 |
| Human salivary amylase | 197 | 233 | 300 |
| Pig pancreas amylase/acarbose | 197 | 233 | 300 |
| | Nucleophile | Acid/base | Acid/base |

In the active site of each of the α-amylases considered, the spermidine molecule was modelled in its original configuration retaining the interactions observed for the AMY1/spermidine complex. All manipulations of the structures (superimposing and insertion of spermidine in the active site) were carried out with the TURBO-FRODO software (Roussel et al., 1991).

Finally, for each of the three structures in which spermidine was inserted, a series energy minimization cycles was carried out.

This energy minimization based on the Powell method was carried out using CNS software version 1.0 (Brünger et al., 1998).

The aim of this stage is the minimization of the energy of the system, firstly, by displacing the atoms which could establish bad contacts between themselves (in particular the water molecules which were not displaced during the insertion of spermidine in the active site), but also to position the atoms in such a way as to maximise the interactions by hydrogen bond between them and therefore move towards the most energetically stable structure model.

Finally, examination of the structure models obtained was carried out with the TURBO-FRODO software.

3) Results:

The models calculated from potential complexes of the above-mentioned 3 α-amylases show that:

firstly, none of the models display bad contacts after fixation of a spermidine molecule in their respective active sites. In fact each amino acid, each water molecule, but also the inserted spermidine are best accommodated within an active site in the process of energy minimisation.

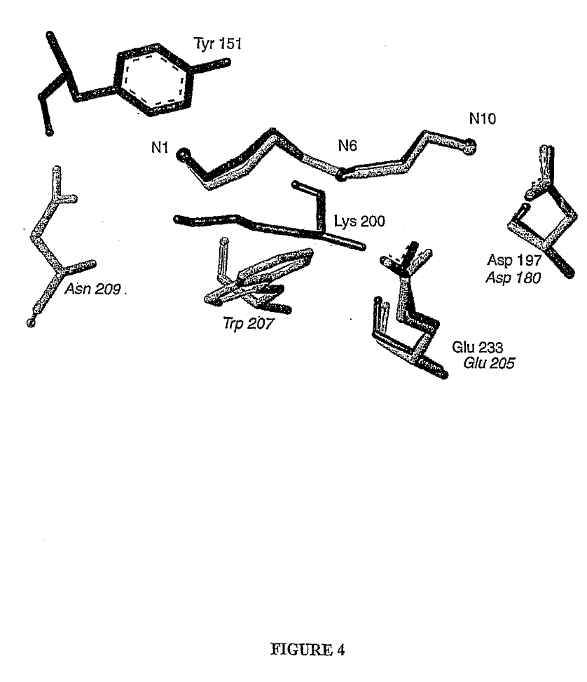

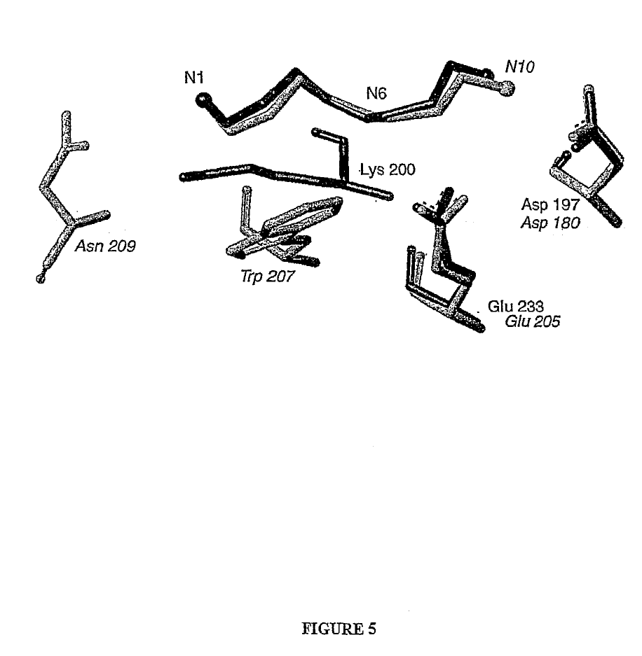

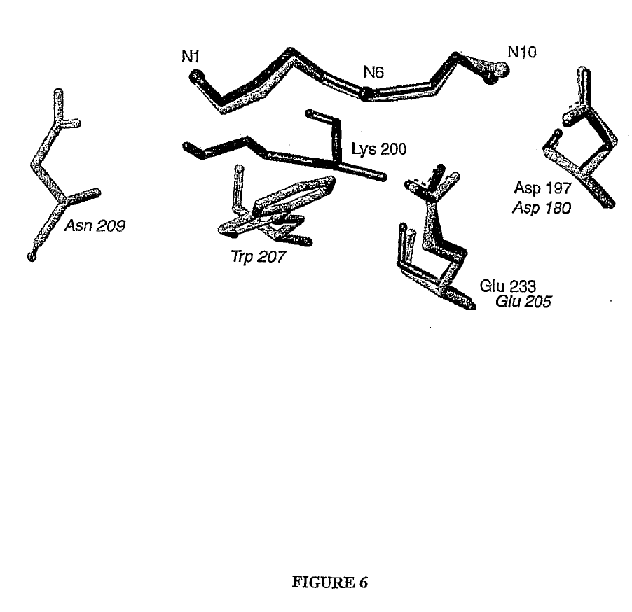

secondly moreover, the formation of numerous hydrogen bonds making it possible to bind and stabilise the spermidine in the active site of the three α-amylases studied can be noted in the models. The direct interactions for these three models are summarized in the tables below (the numbering of the spermidine atoms is the same as in the diagram given above). It is important to note that only first level interactions are given here. Each water molecule mentioned has at least two hydrogen bonds stabilising it. No water molecule mentioned is isolated (see FIGS. 4, 5 and 6).

Table of interactions between spermidine and pig pancreas α-amylase

| Spermidine atoms | Residues or water | Distance (Å) |
|---|---|---|
| N1 | Lys 200 Nζ | 3.4 |
| N1 | Tyr 151 OH | 3.7 |
| N6 | Glu 233 Oε2 | 3.5 |
| N6 | Wat 610 | 3.7 |
| N10 | Asp 197 Oδ1 | 2.8 |
| N10 | Asp 197 Oδ2 | 3.6 |

Table of the interactions between spermidine
and human salivary α-amylase

| Spermidine atoms | Residues or water | Distance (Å) |
|---|---|---|
| N1 | Lys 200 Nζ | 2.8 |
| N1 | Wat 667 | 3.8 |
| N6 | Wat 646 | 2.5 |
| N6 | Wat 586 | 3.7 |
| N10 | Asp 197 Oδ1 | 3.6 |
| N10 | Asp 197 Oδ2 | 3.7 |
| N10 | Glu 233 Oε1 | 3.8 |
| N10 | Wat 563 | 2.4 |
| N10 | Wat 578 | 2.3 |
| N10 | Wat 586 | 3.7 |
| N10 | Wat 604 | 2.7 |

Table of interactions between spermidine
and human pancreatic α-amylase

| Spermidine atoms | Residues or water | Distance (Å) |
|---|---|---|
| N1 | Lys 200 Nζ | 3.5 |
| N1 | Wat 827 | 2.7 |
| N6 | Glu 233 Oε2 | 3.7 |
| N6 | Wat 738 | 2.4 |
| N10 | Asp 197 Oδ1 | 2.9 |
| N10 | Asp 197 Oδ2 | 3.6 |
| N10 | Glu 233 Oε1 | 3.1 |
| N10 | Wat 509 | 2.4 |
| N10 | Wat 594 | 3.8 |
| N10 | Wat 730 | 2.8 |
| N10 | Wat 738 | 2.9 |

4) Discussion and Conclusion:

None of the three amylase-spermidine complex models display bad contacts between the spermidine molecule and the respective enzymes. Therefore, in the active region of the enzyme and during the energy minimisation process, each amino acid residue, each water molecule but also the inserted spermidine is positioned respecting optimal stereochemistry to ensure adequate inhibition of the system. The number of direct hydrogen bonds between the spermidine and a residue or a water molecule is given in the following table for the experimental AMY1/spermidine complex and for the 3 calculated models: pig pancreas α-amylase/spermidine, human salivary α-amylase/spermidine and human pancreatic α-amylase/spermidine.

| Direct hydrogen bonds with | a residue | a water molecule |
|---|---|---|
| AMY1/spermidine | 5 | 4 |
| pig pancreas α-amylase/spermidine | 5 | 1 |
| human salivary α-amylase/spermidine | 4 | 7 |
| human pancreas α-amylase/spermidine | 5 | 6 |

It can thus be noted that spermidine is capable, according to these models, of creating 4 or 5 direct hydrogen bonds with a residue of the studied α-amylases and of creating 1, 6 or 7 hydrogen bonds with water molecules present in the active site, themselves stabilised by other hydrogen bonds.

According to this study, it can be seen for example that the pig pancreas α-amylase/spermidine complex is stabilised by a total of 6 H bonds compared with 11 for human salivary α-amylase/spermidine and human pancreas α-amylase/spermidine models or 9 for the experimental AMY1/spermidine structure.

In this way, these models show the perfect suitability between the spermidine and the active sites of several α-amylases. It is also important that the two most favourable models in terms of active site/spermidine interaction by hydrogen bonds are those of human saliva and pancreas α-amylases. These observations confirm the fact that spermidine or its polyamine derivatives are the molecules of choice for the creation of inhibitors with a human therapeutic use against diabetes and other metabolic disorders such as obesity.

Comparative Inhibition Methods for Spermidine Molecules and Acarbose (GLUCOR®)

Acarbose, a pseudo-tetrasaccharide (a commercially available anti-diabetes medicament (GLUCOR® by Bayer)), binds to amylases to block the active site, as shown with different amylases of various origins, and in particular pig enzyme. FIG. 8 shows an acarbose molecule truncated at the reducing end.(to the right of the figure), after cleavage of the terminal glucose unit.

Similarly, the structure of the isoenzyme 2 of barley α-amylase and acarbose complex was resolved on the atomic scale in our laboratory (Kadziola et al., 1998). In addition, the structure of the isoenzyme 1 of barley α-amylase and acarbose complex was very recently resolved at a resolution of 2.1 Å by X-ray diffraction crystallography.

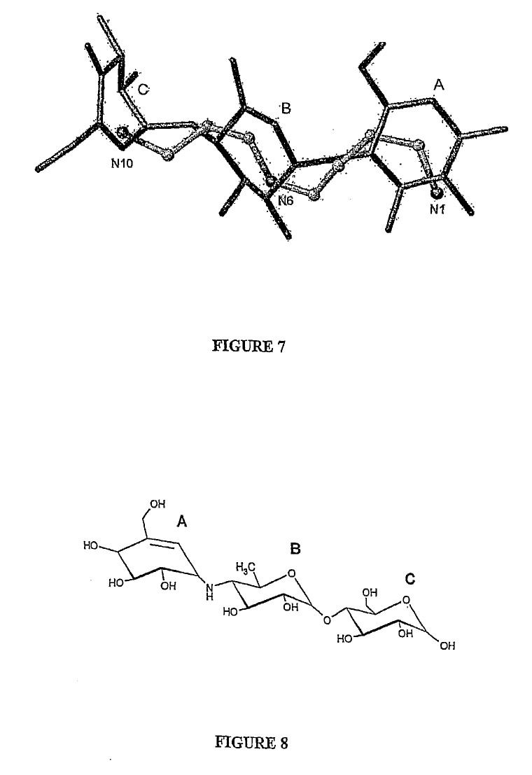

The superimposition of the 3D structures of the isoenzyme 2 of barley α-amylase/acarbose complex and the isoenzyme 1 barley α-amylase/spermidine complex at the level of the active site has made it possible to show the remarkable superimposition of 3 rings of the pseudo-tetrasaccharide acarbose inhibitor and spermidine (see FIG. 7). This result was confirmed by determination of the isoenzyme 1 complex of barley α-amylase/acarbose. This high structural analogy between these inhibitory molecules is also observed in their complexes with mammalian homologous enzymes.

The analogy observed above between the three-dimensional distances of spermidine and acarbose on contact with the same enzyme is quite remarkable. In fact, this structural mimicry is ensured by molecules which are radically different in terms of nature, chemical composition and general properties. They both exert an inhibitory action on α-amylases, and very probably other glycosidases, considering the similarities of the active sites of these different hydrolases. These results also reinforce the possibility of devising polyamine derivatives, such as the glycopolyamines proposed above, for the perfection of therapeutic and pharmacological agents which are effective in the treatment of metabolic disorders linked to the absorption of sugars and/or to disturbances in its regulation (diabetes, obesity, hyperglycemia etc.).

### BIBLIOGRAPHY

Brayer G. D., Y. Luo, et al. (1995). The structure of human pancreatic alpha-amylase at 1.8 A resolution and comparisons with related enzymes. Protein Sci 4(9), 1730-42.

Brünger A. T., Krukowski A. & Erickson J. (1990) Slow-cooling protocols for crystallographic refinement by simulated annealing. Acta Cryst. A 46, 585-593.

Brünger A. T. (1992). X-PLOR, Version 3.1. A system for X-Ray Crystallography and NMR. Yale University Press, New Haven, Conn.

Brünger A. T. (1992). The free R-value: a novel statistical quantity for assessing the accuracy of crystal structures. Nature 355, 472-474.

Brünger A. T., Adams P. D., Clore G. M., Delano W. L., Gros P., Grosse-Kunstleve R. W., Jiang J. -S., Kuszewski J., Nilges M., Pannu N. S., Read R. J., Rice L. M., Simonson T., Warren G. L. (1998). Crystallography & NMR System. Acta Cryst. D 54, 905-921.

Collaborative Computational Project Number 4. (1994). The CCP4 suite: Programs for protein crystallography. Acta Cryst. D 50, 760-763.

Hesse M., Hu W. (1996) Helv. Chim. Acta., 79, 548.

Humora M., Quick J. (1979) J. Org. Chem. 44, 1166).

Kadziola A., Abe J., Svensson B. & Haser R. (1994) Crystal and molecular structure of barley α-amylase. J. Mol. Biol. 239, 104-121.

Kadziola A., Sgaard M., Svensson B. & Haser R. (1998) Molecular structure of a barley α-amylase-inhibitor complex: Implications for starch binding and catalysis. J. Mol. Biol. 278, 205-217.

Kobayashi K., Sumitomo H., Ina Y. (1985) Polymer J. 17, 567.

Laskowski, R. A., MacArthur, M. W., Moss, D. S. & Thornton, J. M. (1993). PROCHECK: a program to check the stereochemistry of protein structures. J. Appl. Cryst. 26, 283-291.

Levchine I., Rajan P., Borloo M., Bollaert W., Haermes A. (1994) Synthesis, 37).

Minor, W. (1993). XDISPLAYF program. Purdue University, West Lafayette, Ind.

Navaza, J. (1994). AmoRe: an automated package for molecular replacement. Acta Cryst. A 50, 157-163.

Otwinoswki, Z. (1993) Oscillation data reduction program. In Proceedings of the CCP4 study weekend: Data Collection and Processing (Sawyer, L., Isaacs, N. and Bailey, S. eds), pp. 56-62, SERC Daresbury Laboratory, England.

Qian, M., R. Haser, et al. (1993). Structure and molecular model refinement of pig pancreatic alpha-amylase at 2.1 Å resolution. J Mol Biol 231(3), 785-99.

Ramasubbu, N., Paloth, V., Luo, Y. G., Brayer, G. D., Levine, M. J. (1996) Structure of human salivary alphα-amylase at 1.6 Å resolution: Implications for its role in the oral cavity. Acta Crystallogr D 52, 435.

Roussel, A. & Cambillau, C. (1991) In Silicon Graphics Geometry Directory 86. Silicon Graphics, Moutain View, Calif.

1. Use of a polyamine derivative or of a polyamine to inhibit the active site of glycosidase enzymes involved in the conversion of polysaccharides to sugars, in particular to glucose, in a living organism.

2. Use of a polyamine derivative or of a polyamine for the preparation of a medicament intended for the diagnosis, the prevention or the treatment of pathologies involving metabolic disorders linked to glycosidases, and more particularly a deregulation of the intestinal absorption of glucose, such as non-insulin dependant diabetes, obesity, hyperglycemia, or hyperlipidemia.

3. Use according to claims 1 to 2 of polyamines, comprising at least 2 positive charges, in particular at least 3 amine functions, and if appropriate at least 1 osidic (or saccharidic) function, linear or branched, the said positive charges, in particular the said amine functions, being spaced out by carbon chains the length of which is approximately 2 carbon atoms to approximately 8 carbon atoms, in particular approximately 3 carbon atoms to approximately 5 carbon atoms.

4. Complex between a polyamine and a glycosidase enzyme, in particular glycosyl hydrolase and more particularly α-amylase, present in all living organisms and responsible for conversion and hydrolysis reactions of oligosaccharides and polysaccharides to simpler osidic molecules such as maltose and glucose, in which the polyamine is fixed at the level of the active site of the enzyme, in particular by hydrogen bonds using the positive charges of the polyamine, corresponding to its amine functions, and the carboxylic functions of lateral chains of amino acids of the above mentioned enzyme, the number of hydrogen bonds being advantageously at least 4.

5. Complex according to claim 4, in which at least two of the sub-sites of the active site of the enzyme are involved in the bond with the above mentioned polyamine.

6. Complex according to any one of claims 4 or 5, in which the enzyme is α-amylase, in particular barley α-amylase (AMY 1) and the following four amino acids of the enzyme: Glu (205), Trp (207), Asn (209), Asp (180) are involved in the bond with the polyamine.

7. Complex according to one of claims 4 to 6, in particular between α-amylase and spermidine comprising at least one of the following interactions, and in particular all of the following interactions, which are hydrogen type bonds and which can be defined as indicated hereafter:

| Spermidine atoms: | AMY1 residues or water: | Distance (Å): |
|---|---|---|
| Number of the nitrogen atom | | |
| N1 | W207 Nε1 | 3.7 |
| N1 | N209 Oδ1 | 3.4 |
| N1 | Wat 1250 | 3.1 |
| N6 | E205 Oε2 | 3.7 |
| N10 | D180 Oδ1 | 2.7 |
| N10 | D180 Oδ2 | 3.7 |
| N10 | Wat 1087 | 2.8 |
| N10 | Wat 1102 | 2.9 |
| N10 | Wat 1259 | 2.8 |

8. Complex according to one of claims 4 to 6, in which the polyamine is a chain of approximately 6 to approximately 20 atoms, in particular comprising approximately 6 to 15, and advantageously approximately 6 to 10 nitrogen atoms, advantageously two primary amine functions, at each of the ends of the polyamine respectively, at least one of the amine functions being optionally substituted by a substituent chosen from the linear or cyclic polysaccharides having, approximately 1 to approximately 6 osidic units which are advantageously glucose, maltose, or cyclodextrine, and at least one of the nitrogen atoms inside the chain being optionally substituted by a substituent chosen from the oligosaccharides, linear or cyclic, with 1 to 6, in particular 1 to 3 osidic units, in particular glucose, maltose or cyclodextrine.

9. Complex according to one of claims 4 to 8, according to which
the enzyme is α-amylase and
the polyamine is chosen from spermidine and its derivatives, said polyamine corresponding to one of the following general formulae: 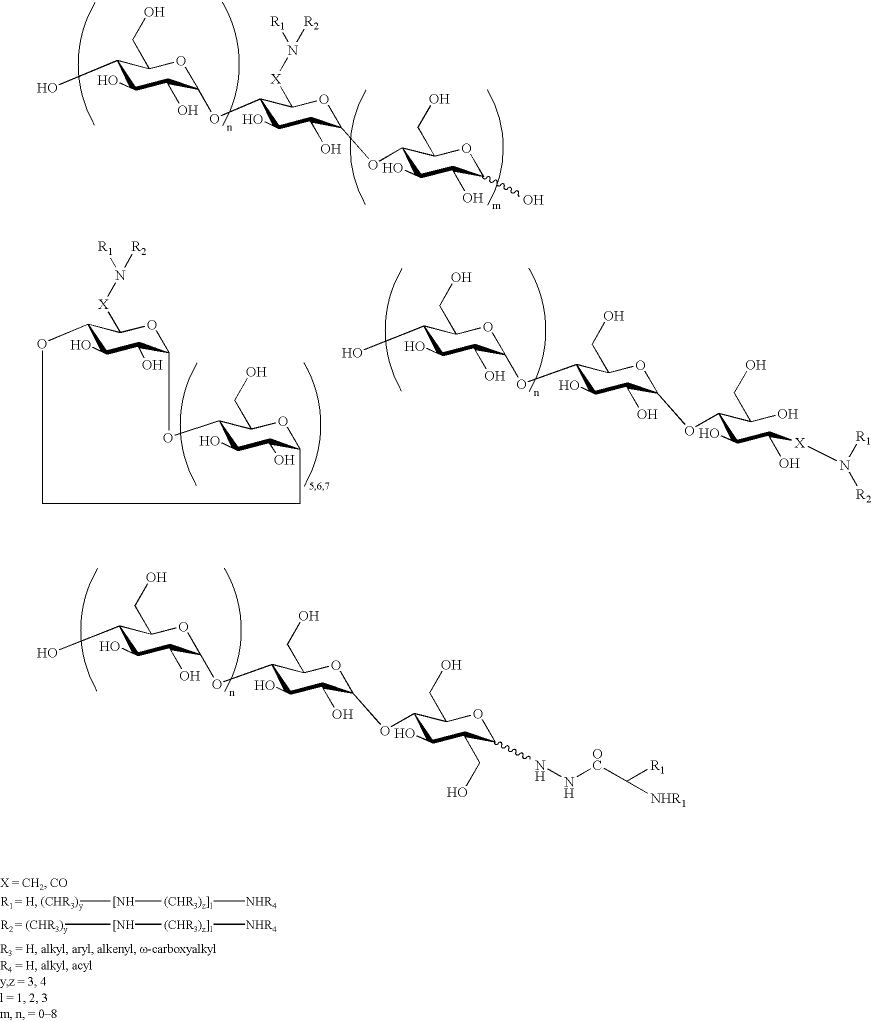
or corresponding, in an advantageous manner, to this general formula:
NH₂—(CH₂)ₘ—NH—(CH₂)ₙ—NH—R
in which m and n are comprised between 2 and 8, and in which the R group is either H, or a glucose group, or a maltooligosaecharide group, or an aryl group advantageously comprising 6 carbon atoms, or an alkyl group advantageously comprising 6 carbon atoms,
or advantageously to the following formulae:
NH₂(CH₂)₃NH(CH₂)₃NH₂
NH₂(CH₂)₄NH(CH₂)₄NH₂
NH₂(CH₂)₄N(CH₃)(CH₂)₃NH₂
NH₂(CH₂)₄NH CH₂CH(CH₃)CH₂NH₂
NH₂(CH₂)₄NH (CH₂)₂CH(CH₃)NH₂
NH₂(CH₂)₃CH(CH₃)NH(CH₂)₃NH₂ 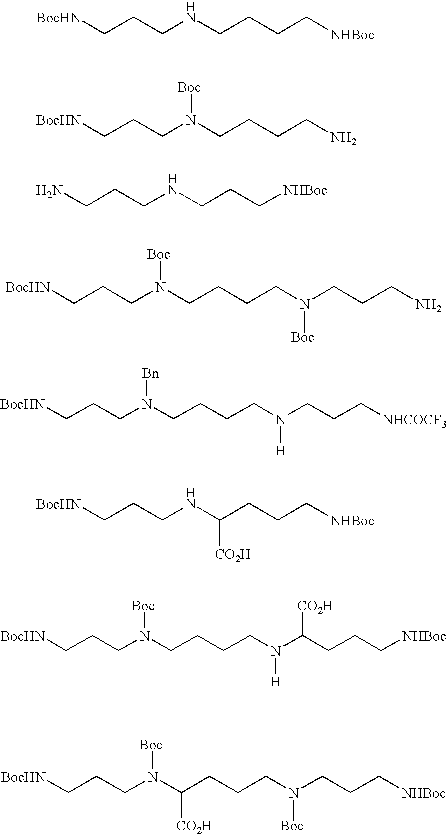
Bn=benzyl
Boc=butyloxycarbonyl,
the length of the alkyl chains between the nitrogen atoms being able to vary from approximately 2 to approximately 8 carbons, and in particular from approximately 3 to approximately 5 carbons, the alkyl chains being also able to be substituted by chemical groups preferably comprising an aminated function or derivative, the alkyl chains being also able to comprise nitrogen atoms other than those represented in the formulae above.

10. Complex according to one of claims 4 to 9, having the following crystallographic characteristics:
crystalline parameters of the elementary lattice:
a=42.6 Å
b=80.6 Å
c=137.0 Å
α=β=γ=90.0°
orthorhombic crystalline system
space group P2₁2₁2 ₁
number of enzyme molecules by crystalline lattice of the elementary lattice=4

11. Polyamine capable of being used in the constitution of the complex according to any one of claims 4 to 9, and in particular constituted by a chain of approximately 6 to approximately 20 atoms, in particular comprising approximately 6 to approximately 15, and advantageously approximately 6 to approximately 10 nitrogen atoms, advantageously two primary amine functions, at each of the ends of the polyamine respectively, at least one of the amine functions being optionally substituted by a substituent chosen from the linear or cyclic polysaccharides with approximately 1 to approximately 6 osidic units advantageously glucose, maltose, or cyclodextrine and at least one of the nitrogen atoms inside the chain being optionally substituted by a substituent chosen from the linear or cyclic oligosaccharides with 1 to 6 units, in particular 1 to 3 osidic units, in particular glucose, maltose or cyclodextrine on condition that the polyamine is different from the following products:
spermidine
spermine

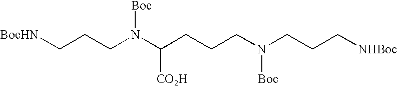

12. Polyamine derivatives according to claim 11, corresponding to one of the following general formulae: 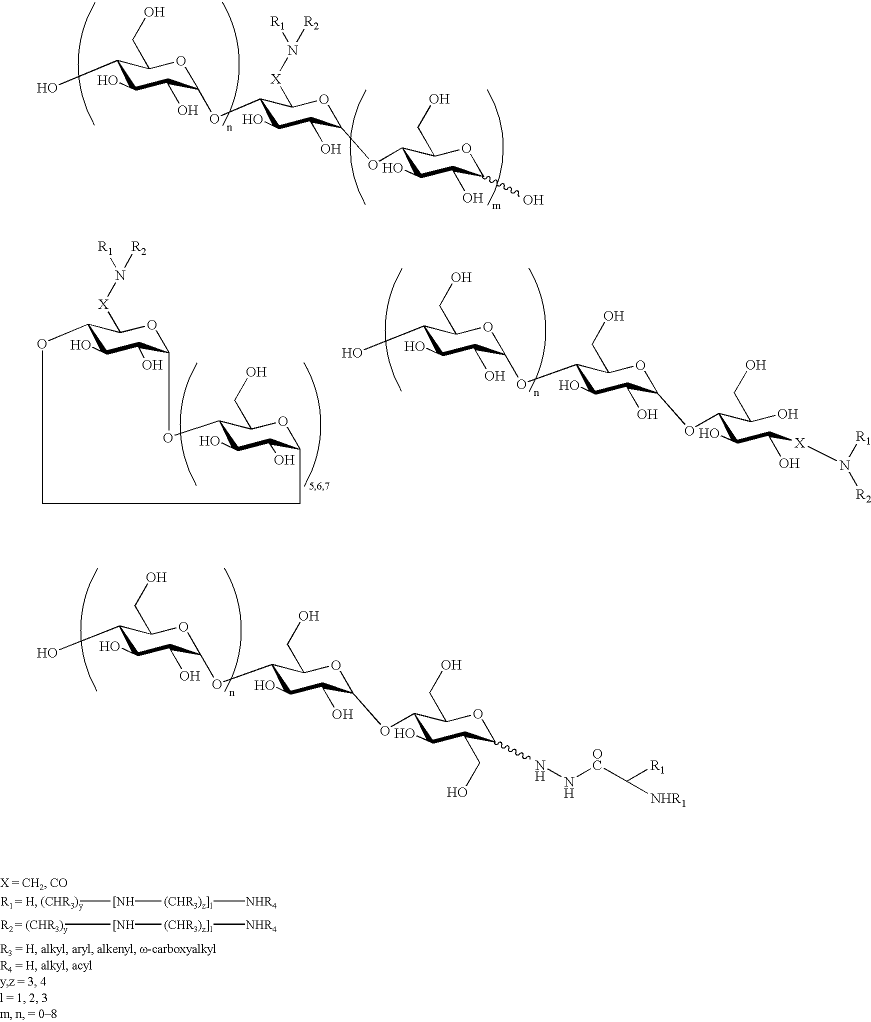
or corresponding, in an advantageous manner, to this general formula:
NH₂—(CH₂)ₘ—NH—(CH₂)ₙ—NH—R
in which m and n are comprised between 2 and 8, and in which the R group is either H, or a glucose group, or a maltooligosaccharide group, or an aryl group advantageously comprising 6 carbon atoms, or an alkyl group advantageously comprising 6 carbon atoms,
or to one of the following formulae: 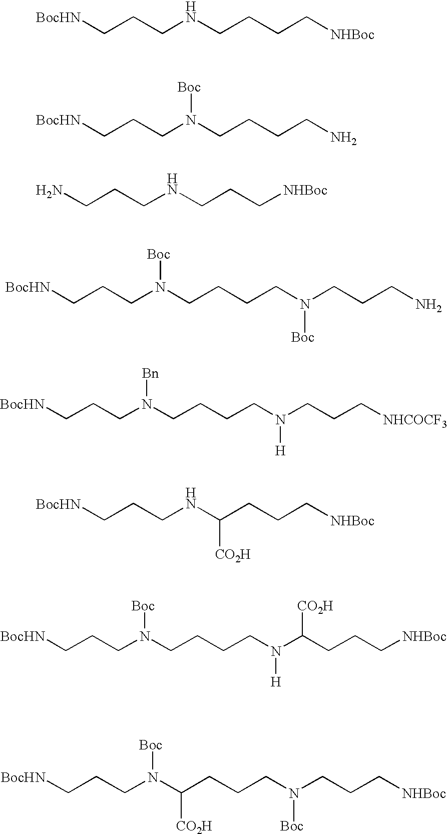
Bn=benzyl
Boc=butyloxycarbonyl,
the length of the alkyl chains between the nitrogen atoms being able to vary from approximately 3 to approximately 5 carbon atoms, the alkyl chains being also able to be substituted by chemical groups preferably comprising an aminated function or derivative, the alkyl chains being also able to comprise nitrogen atoms other than those represented in the formulae above.
